(19) United States
(12) Patent Application Publication
(10) Pub. No.: US 2005/0118102 A1
(43) Pub. Date: Jun. 2, 2005

(54) Spin resonance heating and/or imaging in medical applications

(75) Inventors: Xiao Dong Xiang, Danville, CA (US); Gang Wang, Albany, CA (US); Haitao Yang, Albany, CA (US); Gerald Dionne, Winchester, MA (US); Ted Sun, Fremont, CA (US)

Correspondence Address: QUINE INTELLECTUAL PROPERTY LAW GROUP, P.C., P O BOX 458, ALAMEDA, CA, 94501, US

(73) Assignee: Intematix Corporation

(21) Appl. No.: 10/835,247

(22) Filed: Apr. 28, 2004

Related U.S. Application Data
(60) Provisional application No. 60/466,099, filed on Apr. 28, 2003.

(51) Int. Cl. A61K 49/00

(52) U.S. Cl. 424/9.34; 530/391.1

(57) ABSTRACT

This invention pertains to the use of spin resonance absorption heating as a therapeutic treatment method. It was a surprising discovery that electron spin resonance absorption of superparamagnetic (SPM) nanoparticles can be used as a heating method, more preferably as an in vivo heating method that can be utilized in a variety of therapeutic contexts.

### CROSS-REFERENCE TO RELATED APPLICATIONS

This application claims benefit of and priority to U.S. Ser. No. 60/466,099, filed on Apr. 28, 2003, which is incorporated herein by reference in its entirety for all purposes.

### STATEMENT AS TO RIGHTS TO INVENTIONS MADE UNDER FEDERALLY SPONSORED RESEARCH AND DEVELOPMENT

Not Applicable

### FIELD OF THE INVENTION

This invention is related to a method of imaging and/or selective heating therapy using nano-sized superparamagnetic (SPM) particles. In certain embodiments, the SPM particle can be physically delivered to particular cells or tissues, or chemically coated to target particular cells or molecules. Upon application of an RF field in magnetic field, the particle can absorb the RF power by magnetic resonance and the energy is released as heat, which can selectively destroy the targeted cells or tissues adjacent to the particles. Magnetic field gradient can also be used to localize the heating region, to a smaller region than the region than particles are distributed.

### BACKGROUND OF THE INVENTION

Electromagnetic radiation (e.g. X-ray and γ-ray from radioactive elements) with very high-energy photon particles has been traditionally used for the therapeutic treatment of certain diseases such as cancer. The high-energy radiation beam can be focused to a specific location, even deep within the body, to destroy the targeted cells. However, the normal cells at the same location will also simultaneously be killed. Moreover, the cells in other regions in the beam path can also be killed or damaged depending on the intensity of the photons at any particular location. Adverse effects may follow irradiation therapy; among which the more common are disturbances of bone growth, cartilage abnormalities, scoliosis and radiation osteonecrosis (The Encyclopaedia of Medical Imaging Volume III:1). Consequently, there is always a conflict between the dosages that will effectively kill the disease cells and keep enough normal cells for recovery.

It is highly desirable if the radiation can be specifically targeted only to diseased cells at specific locations. It is also desirable that the radiation energy and dosage can be dramatically lowered for safety reasons. Consequently hyperthermia has been explored as a treatment tool for cancers, for other pathologies treated by inhibiting cell growth or proliferation, and for the cosmetic ablation of tissues.

It is known that elevating the temperature of tumors is helpful in the treatment and management of cancerous tissues. The mechanisms of selective cancer cell eradication by hyperthermia are not completely understood. Four cellular effects of hyperthermia on cancerous tissue have been proposed, (i) changes in cell or nuclear membrane permeability or fluidity, (ii) cytoplasmic lysomal disintegration, causing release of digestive enzymes, (iii) protein thermal damage affecting cell respiration and the synthesis of DNA or RNA and (iv) potential excitation of immunologic systems.

Treatment methods for applying heat to tumors include the use of direct contact radio-frequency (RF) applicators, microwave radiation, inductively coupled RF fields, ultrasound, and a variety of simple thermal conduction techniques. Among the problems associated with all of these procedures is the requirement that highly localized heat be produced at depths of several centimeters beneath the surface of the body. Certain techniques have been developed with microwave radiation and ultrasound to focus energy at various desired depths. RF applications may be used at depth during surgery. However, the extent of localization is generally poor, with the result that healthy tissue may be harmed. Induction heating gives rise to poor localization of the incident energy as well. Although induction heating may be achieved by placing an antenna on the surface of the body, superficial eddy currents are generated in the immediate vicinity of the antenna, when it is driven using RF current, and unwanted surface heating occurs with little heating delivered to the underlying tissue.

### SUMMARY OF THE INVENTION

In order to overcome the shortcomings of previously described techniques, this invention provides electron spin resonance heating methods for biomedical applications. Magnetic resonance (e.g., MRI) methods and nuclear spin resonance (e.g. NMR) methods have been proposed for hyperthermic treatment modalities. Spin resonance heating occurs when applied radiation field (microwave or RF) frequency, magnetic field and material's gyromagnetic ratio satisfy the following equation (Poole (1983) Electron Spin Resonance (2nd Edition), A Wiley-Interscience Pub.):
hv=gμ_{B}B 1
where h is Plank constant, v the magnetic spin resonant frequency, B the external magnetic field, g the gyromagnetic ratio, and μ_{B} is Bohr magneton for electron spin resonance (ESR); for nuclear magnetic resonance (NMR), μ_{B} should be replaced by nuclear magneton μ_{N}. Nuclear spins or electron spins absorb photon energy at the spin resonance and jump to higher energy level precessing coherently. As the spin precessing relaxes through spin-lattice interaction, the absorbed electromagnetic energy turns into heat. The heat generation is proportional to the density of un-paired spin and spin population difference. The spin population difference in the two adjacent Zeeman levels is governed by Boltzmann statistics:

$$Δ n = 1 - exp ( - \frac{hv}{kT} ) \qquad 2$$

Since the energy difference between two Zeeman levels is small, at elevated temperatures, thermal excitation makes the spins almost equally occupy both energy levels leaving a very small fraction of spin to contribute to the spin resonance. At room temperature and in a 5 T magnetic field; this corresponds to a factor of 10⁻⁵ reduction in resonance absorption for a typical NMR (or MRI). Therefore, nuclear spin resonance absorption is generally not effective in generating heat. In addition, nuclear spin resonance absorption heats up all protons, which may not be suitable for targeted therapeutic treatment.

Since the mass of an electron is about 1863 times smaller than a proton for electron spin resonance (ESR), the spin population difference for ESR is approximately 10⁻² at room temperature and a 5 T magnetic field. The required frequency for electromagnetic radiation to excite the spin resonance using traditional approaches is much higher than that of NMR, typically 9.8 GHz. The radiation at this frequency cannot penetrate deeply and suffers a large dielectric loss in biological tissues that renders the technique useless in most cases. Recently, there has been much effort put forth to use lower frequency ESR technique (200 MHz to 3 GHz) for MRI. However, if we lower the frequency of ESR to the same frequency of NMR, it will have the same reduction of 10⁻⁵ in spin population difference, and therefore, resonance absorption the same as NMR.

This invention pertains to the use of spin resonance absorption heating as a therapeutic treatment method. It was a surprising discovery that electron spin resonance absorption of superparamagnetic (SPM) nanoparticles can be used as an effective heating method, more preferably as an in vivo heating method that can be utilized in a variety of therapeutic contexts.

The superparamagnetic nanoparticles can be attached to targeting moieties that specifically/preferentially bind to a desired target cell, tissue, organ, etc. thereby allowing selective heating of the target. Spatially resolved (localized) heating can also be provided by tailoring the magnetic field gradient during electron spin resonance (ESR) as described herein. Since spin resonance occurs only when the applied magnetic field and electromagnetic radiation energy satisfy certain resonance conditions, heating can be directed and limited only to the SPM particles at a specific location. As a result, only cells, and/or tissues, and/or organs, etc., adjacent to the spatially selected particles will be heated and, if desired, damaged. Most of the normal cells will not be affected during the treatment.

In certain embodiments, mechanism, superparamagnetic particles are coated with certain inorganic or organic chemicals and linked to antibodies or other targeting moieties in order for them to be attached to the targeted biological specimen, such as a disease cell, a viruses, or other pathogen, etc. Because of the long-range spin-spin correlation in superparamagnetic materials, the spin population difference is nearly one in contrast to that in nuclear or electron paramagnetic spin resonance where the spin population difference is only 10⁻⁵. This makes resonance absorption at least 5 orders of magnitude higher than conventional NMR or ESR. As a consequence, spin resonance heating will be 5 orders of magnitude more effective and viable to realistic therapeutic applications. Since the superparamagnetic spin resonance is far away from the spin resonance of any cells in biological specimen under the same magnetic field, the absorption and conversion of electromagnetic energy to heat is highly selective only to the resonating SPM particles and the immediate vicinity. The other regions of the subject (e.g., a human body can be spared of any harmful side effects.

Thus, in one embodiment, this invention nprovides composition for selectively heating (via electron spin resonance (ESR) and/or imaging a cell, tissue, or organism. The composition comprises a superparamagnetic nanoparticle attached to a targeting moiety that specifically binds to a biological target comprising the cell tissue, or organ. The superparamagnetic nanoparticle comprises a material that typically has an electron spin resonance (ESR) Q greater than 10, more preferably greater than 50 or 100, and most preferably greater than about 500. In certain embodiments, Q ranges from about 10 to 3000, more preferably from about 100 to about 1000. In certain embodiments, the superparamagnetic nanoparticle comprises a garnet or a spinel (e.g., a garnet or a spinel selected from Table 2. In certain embodiments, the superparamagnetic nanoparticle comprises yttrium ion garnet (YIG), more preferably substituted YIG (e.g. as shown in Table 2, or with aluminum, gallium, indium, ferrite, etc.). In certain embodiments, the superparamagnetic nanoparticle comprises gamma-Fe2O3. The targeting moiety can be directly attached to the superparamagnetic nanoparticles or attached to the sperparamagnetic nanoparticle through a linker. In various embodiments, the superparamagnetic nanoparticle (SNP) is attached to a single targeting moiety, the SNP is attached to a plurality of targeting moieties, or a plurality of SNPs are attached to a single targeting moiety. Suitable targeting moieties include, but are not limited to a protein, an antibody, a lectin, a saccharide, a vitamin, a steroid, a steroid analogue, a hormone, a nucleic acid, and the like. In certain embodiments, the cell or tissue is a cancer cell. In certain embodiments, the biological target is a cancer marker (e.g., Caf-1, C-myc, p53, Ki67, Her2, Her4, BRCA1, BRCA2, Lewis Y (LeY), CA 15-3, G250, HLA-DR cell surface antigen, CEA, CD20, CD22, integrin, cea, 16, EGFr, AR, PSA, other growth factor receptors, and the like). In certain embodiments, the SPN has at least one dimension less than about 500 nm, in certain embodiments, the SPN has no dimension greater than about 500 nm, and in certain embodiments, SPN has at least one dimension less than about 100 nm.

In another embodiment, this invention provides a composition for selectively heating or imaging a cell, tissue, or organ. The composition typically comprises superparamagnetic nanoparticles (e.g., any of the SPNs as described above) in a pharmacologically acceptable excipient.

Also provided is a method of selectively heating an organ, a cell, a tissue, a molecule, etc. The method typically involves contacting the cell, tissue, or molecule with a composition comprising a superparamagnetic nanoparticle (SPN) attached to a targeting moiety that specifically binds to a biological target comprising the cell, tissue, or molecule; and heating the superparamagnetic nanoparticle using electron spin resonance. In certain embodiments, the electron spin resonance is at an RF ranging from about RF frequency ranging from about 200 to about 2,000 MHz MHz. In certain embodiments, the electron spin resonance is at an RF ranging from about 500 to about 1,000 MHz. In certain embodiments, the electron spin resonance is spatially localized by a magnetic field gradient over a region smaller than the region over which the superparamagnetic nanoparticles are distributed. The SPN attached to a targeting moiety includes, but is not limited to any of the SPNs attached to targeting moieties described above.

In still another embodiment, this invention provides a selectively heating a cell, tissue, or organ. The method typically involves delivering a plurality of superparamagnetic nanoparticles to a location adjacent to or contacting the cell, tissue, or organ; and heating the superparamagnetic nanoparticles using electron spin resonance. The method can be performed ex vivo, in vivo, and in situ. In certain embodiments, the superparamagnetic nanoparticles are delivered directly into the cell, tissue, or organ (e.g., by injection, via a catheter, during a surgical procedure, etc.). In certain embodiments, the superparamagnetic nanoparticles are delivered systemically administered to an organism. The SPNs include, but are not limited to any of the SPNs described above. In certain embodiments, the electron spin resonance is at an RF ranging from about 200 to about 2,000 MHz. In certain embodiments, the electron spin resonance is at an RF ranging from about 500 to about 1,000 MHz. The electron spin resonance can be spatially localized by a magnetic field gradient over a region smaller than the region over which the superparamagnetic nanoparticles are distributed. The method can, optionally, further involve imaging the cell, tissue, organ, or molecule (e.g., via thermography, MRI, ESR, x-ray, etc.). In various embodiments, the cell or tissue is a cancer cell.

In certain embodiments, this invention provides methods of selectively heating a cancer cell. The methods typically involve contacting a cancer cell with a chimeric molecule comprising a targeting moiety attached to an epitope tag, where the targeting moiety specifically binds to a cancer cell; contacting the chimeric molecule with a superparamagnetic nanoparticle attached to a binding moiety (directly or through a linker) that specifically binds to the epitope tag thereby associating said superparamagnetic nanoparticle with said cancer cell; and performing electron spin resonance to heat the superparamagnetic nanoparticle. Suitable superparamagnetic nanoparticles (SPNs) are SPNs for electron spin resonance and include, but are not limited to any of the SPNs described herein (e.g., SPNs with a Q greater than 10, SPNs comprising a material in Table 2, etc.). The method can, optionally, further comprise imaging the cell, tissue or molecule preferably by detecting the SPN, e.g., via thermography, MRI, ESR, x-ray, etc. In certain embodiments, the chelate comprises DOTA. Suitable epitope tags include, but are not limited to avidin or a biotin. In certain embodiments, the targeting moiety is selected from the group consisting of a protein, an antibody, a lectin, a saccharide, a vitamin, a steroid, a steroid analogue, a hormone, a nucleic acid, and an antibody. In certain embodiments, the targeting moiety specifically or preferentially binds a cancer marker selected from the group consisting of Caf-1, C-myc, p53, Ki67, Her2, Her4, BRCA1, BRCA2, Lewis Y (LeY), CA 15-3, G250, HLA-DR cell surface antigen, CEA, CD20, CD22, integrin, cea, 16, EGFr, AR, PSA, and other growth factor receptors.

This invention also provides kits for kit for selectively heating (e.g., via ESR) or imaging a cell, tissue, organ, etc. The kit typically includes a container containing a superparamagnetic nanoparticle (SPN) attached to a targeting moiety that specifically binds to a biological target comprising the cell or tissue. The SPN attached to a targeting moiety includes, but is not limited to any of the SPNs attached to targeting moieties described above. The SPN attached to a targeting moiety can be provided dried or suspended in a solution (e.g., a pharmacologically acceptable excipient).

In another embodiment, a kit is provided for selectively heating or imaging a cell or tissue. The kit typically includes a container containing a superparamagnetic nanoparticle where the nanoparticle is derivatized for coupling to a targeting moiety. The kit can, optionally, further comprising a targeting moiety for attaching to the superparamagnetic nanoparticles, and/or instructional materials teaching the use of the superparamagnetic nanoparticles to selectively heat or image a cell or tissue. In certain embodiments, the targeting moiety is a moiety that specifically binds to a cancer cell.

Definitions

The term “nanoparticle”, as used herein refers to a particle having at least one dimension equal to or smaller than about 500 nm, preferably equal to or smaller than about 100 nm, more preferably equal to or smaller than about 50 or 20 nm, or having a crystallite size of about 10 nm or less, as measured from electron microscope images and/or diffraction peak half widths of standard 2-theta x-ray diffraction scans.

The term “specifically binds”, as used herein, when referring to a targeting moiety and its target refers to a binding reaction that is determinative of the presence of the target in a heterogeneous population of molecules (e.g., proteins and other biologics). Thus, under designated conditions (e.g. binding assay conditions in the case of antibody or stringent hybridization conditions in the case of a nucleic acid), the specified targeting moiety preferentially binds to its particular “target” molecule and preferentially does not bind in a significant amount to other molecules present in the sample. In certain embodiments, the terms “specific binding” or “preferential binding” refer to that binding which occurs between such paired species as enzyme/substrate, receptor/agonist, antibody/antigen, and lectin/carbohydrate which may be mediated by covalent and/or non-covalent interactions. When the interaction of the two species typically produces a non-covalently bound complex, the binding which occurs is typically electrostatic, and/or hydrogen-bonding, and/or the result of lipophilic interactions. Accordingly, “specific binding” occurs between pairs of species where there is interaction between the two that produces a bound complex. In particular, the specific binding is characterized by the preferential binding of one member of a pair to a particular species as compared to the binding of that member of the pair to other species within the family of compounds to which that species belongs.

The terms “targeting moiety”, as used herein, refers generally to a molecule that binds to a particular target molecule and forms a bound complex as described above. The binding can be highly specific binding, however, in certain embodiments, the binding of an individual targeting moiety to the target molecule can be with relatively low affinity and/or specificity. The ligand and its corresponding target molecule form a specific binding pair. Examples include, but are not limited to small organic molecules, sugars, lectins, nucleic acids, proteins, antibodies, cytokines, receptor proteins, growth factors, nucleic acid binding proteins and the like which specifically bind desired target molecules, target collections of molecules, target receptors, target cells, and the like.

The term “cancer marker” refers to biomolecules such as proteins that are useful in the diagnosis and prognosis of cancer. As used herein, “cancer markers” include but are not limited to: PSA, human chorionic gonadotropin, alpha-fetoprotein, carcinoembryonic antigen, cancer antigen (CA) 125, CA 15-3, CD20, CDH13, CD 31, CD34, CD105, CD146, D16S422HER-2, phospatidylinositol 3-kinase (PI 3-kinase), trypsin, trypsin-1 complexed with alpha(1)-antitrypsin, estrogen receptor, progesterone receptor, c-erbB-2, bcl-2, S-phase fraction (SPF), p185erbB-2, low-affinity insulin like growth factor-binding protein, urinary tissue factor, vascular endothelial growth factor, epidermal growth factor, epidermal growth factor receptor, apoptosis proteins (p53, Ki67), factor VIII, adhesion proteins (CD-44, sialyl-TN, blood group A, bacterial lacZ, human placental alkaline phosphatase (ALP), alpha-difluoromethylornithine (DFMO), thymidine phosphorylase (dTHdPase), thrombomodulin, laminin receptor, fibronectin, anticyclins, anticyclin A, B, or E, proliferation associated nuclear antigen, lectin UEA-1, cea, 16, and von Willebrand's factor.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term also includes variants on the traditional peptide linkage joining the amino acids making up the polypeptide.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10): 1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111 :2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The term “biotin” refers to biotin and modified biotins or biotin analogues that are capable of binding avidin or various avidin analogues. “Biotin”, can be, inter alia, modified by the addition of one or more addends, usually through its free carboxyl residue. Useful biotin derivatives include, but are not limited to, active esters, amines, hydrazides and thiol groups that are coupled with a complimentary reactive group such as an amine, an acyl or alkyl group, a carbonyl group, an alkyl halide or a Michael-type acceptor on the appended compound or polymer.

Avidin, typically found in egg whites, has a very high binding affinity for biotin, which is a B-complex vitamin (Wilcheck et al. (1988) Anal. Biochem, 171: 1). Streptavidin, derived from Streptomyces avidinii, is similar to avidin, but has lower non-specific tissue binding, and therefore often is used in place of avidin. As used herein “avidin” includes all of its biological forms either in their natural states or in their modified forms. Modified forms of avidin which have been treated to remove the protein's carbohydrate residues (“deglycosylated avidin”), and/or its highly basic charge (“neutral avidin”), for example, also are useful in the invention.

The term “residue” as used herein refers to natural, synthetic, or modified amino acids.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker. Huston, et al. (1988) Proc. Nat. Acad. Sci. USA, 85: 5879-5883. While the V_{H} and V_{L} are connected to each as a single polypeptide chain, the V_{H} and V_{L} domains associate non-covalently. The first functional antibody molecules to be expressed on the surface of filamentous phage were single-chain Fv's (scFv), however, alternative expression strategies have also been successful. For example Fab molecules can be displayed on phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to, e.g., g3p (see, e.g., U.S. Pat. No. 5,733,743). The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778). Particularly preferred antibodies should include all that have been displayed on phage (e.g., scFv, Fv, Fab and disulfide linked Fv (Reiterii et al. (1995) Protein Eng. 8: 1323-1331).

The terms “epitope tag” or “affinity tag” are used interchangeably herein, and usually refers to a molecule or domain of a molecule that is specifically recognized by an antibody or other binding partner. The term also refers to the binding partner complex as well. Thus, for example, biotin or a biotin/avidin complex are both regarded as an affinity tag. In addition to epitopes recognized in epitope/antibody interactions, affinity tags also comprise “epitopes” recognized by other binding molecules (e.g. ligands bound by receptors), ligands bound by other ligands to form heterodimers or homodimers, HiS₆ bound by Ni-NTA, biotin bound by avidin, streptavidin, or anti-biotin antibodies, and the like.

Epitope tags are well known to those of skill in the art. Moreover, antibodies specific to a wide variety of epitope tags are commercially available. These include but are not limited to antibodies against the DYKDDDDK (SEQ ID NO:1) epitope, c-myc antibodies (available from Sigma, St. Louis), the HNK-1 carbohydrate epitope, the HA epitope, the HSV epitope, the His₄, His₅, and His₆ epitopes that are recognized by the His epitope specific antibodies (see, e.g., Qiagen), and the like. In addition, vectors for epitope tagging proteins are commercially available. Thus, for example, the pCMV-Tag1 vector is an epitope tagging vector designed for gene expression in mammalian cells. A target gene inserted into the pCMV-Tag1 vector can be tagged with the FLAG® epitope (N-terminal, C-terminal or internal tagging), the c-myc epitope (C-terminal) or both the FLAG (N-terminal) and c-myc (C-terminal) epitopes.

A PEG type linker refers to a linker comprising a polyethylene glycol (PEG).

### BRIEF DESCRIPTION OF THE DRAWINGS

FIG. 1 illustrates an instrument set-up used for characterization of particle spin resonance detection and heating.

FIG. 2 illustrates ferromagnetic resonance of YIG sphere measured by EMP.

FIGS. 3A and 3B illustrate RF coil and protection circuit design. FIG. 3A: Surface R.F. coil, which is tuned to resonance with the tuning capacitor CT and matched to 50 ohms with a matching capacitor CM. FIG. 3B: Circuit diagram for receiver isolation using a quarter wavelength cable and protection Zener diode.

FIGS. 4A and 4B illustrate particle surface derivatization (FIG. 4A) and cell surface recognition (FIG. 4B).

FIGS. 5A and 5B illustrate functionalization of a superparamagnetic nanoparticles for attaching a targeting moiety. FIG. 5A illustrates silanization to introduce an amino (—NH₂) functional group on a nanoparticle surface. FIG. 5B illustrates direct protein immobilization on —NH₂ functionalized nanoparticles through activation by glutaraldehyde

FIGS. 6A and 6B illustrate generation of the magnetic field gradient. FIG. 6A: The x gradient is formed by a current that runs on a cylinder such that the two arcs above are both bringing current around the cylinder in a clockwise direction. The arcs shown below will bring current around the cylinder in a counter-clockwise direction. This creates a magnetic field pointing in the z direction that varies in strength along the x direction. For a y gradient, this configuration need only be rotated by 90°. FIG. 6B: A magnetic field gradient in the z direction is made by two circular coils whose currents run in opposite directions. This makes a magnetic field that points in the z direction and varies in strength along z.

### DETAILED DESCRIPTION

This invention pertains to the surprising discovery that electron spin resonance can be used for effective and local heating of superparamagnetic particles, preferably superparamagnetic nanoparticles in, or adjacent to, biological specimens (e.g., cells, tissues, organs, organisms, etc.). The local heating obtainable using the methods described herein is effective in the hyperthermic (e.g., thermal ablation, temperature-induced apoptosis, etc.) treatment of cancers (or other conditions characterized by cellular hyperproliferation), the cosmetic ablation of tissues, and the like.

A high degree of specificity can be achieved using one or both of two approaches. In the first approach, the superparamagnetic nanoparticles are coupled to a targeting moiety that specifically binds to the target cell, tissue, organ, etc. thereby preferentially localizing the nanoparticles at the target site where under the appropriate field conditions, they deliver heat to the target. In the second approach, heating can be localized by the configuration of the magnetic field used in the procedure. Thus, the nanoparticles need not be attached to targeting moieties and extremely local heating can be achieved by the use of appropriate field gradients as described herein. In certain embodiments, specificity is achieved both by the use of targeting moieties and specific tailoring of the field gradients.

The method of this invention are particularly well suited for therapeutic applications because they also permit visualization, preferably non-invasive visualization of the superparamegnetic particles and thereby of the cells, tissues, organs, etc. that are tagged by and/or associated with the nanoparticles. Visualization methods include, but are not limited to X-rays (the nanoparticles can act as contrast agents), magnetic resonance imaging (MRI), electron spin resonance imaging, thermographic imaging (e.g., by detecting the signature of the heated nanoparticles), and the like. In various embodiments, the visualization can be performed simultaneously or independently of the particle heating.

Superparamagnetic particles are magnetic materials (e.g., ferromagnetic materials, ferromagnetic materials, etc.) with essentially zero magnetic coercively or spontaneous magnetization. At a zero applied magnetic field, the particles do not manifest magnetization and exert magnetic force on each other. In a non-zero magnetic field, due to long-range coupling of electron spins in the superparamagnetic materials, the spins align along the direction of the applied magnetic field. As a result, the spin population difference is nearly one below Curie temperature. Therefore, this approach provides the highest possible spin resonance absorption efficiency and can provide a significant and useful heating effect even at radio frequencies. At radio frequencies, the radiation can penetrate deep into a biological specimen, including human and animal, without heating up the other cells or tissues since these frequencies are far away from the water molecule absorption frequency spectrum.

A 3-Dimensional gradient configuration of magnetic field can be easily used to select specific locations that satisfy the equation (1) for spin resonance absorption heating. Importantly, superparamagnetic spin resonance imaging (with reduced RF frequency radiation) can be performed with the same equipment before, during, or after the heating therapy is performed. The required magnetic field is much lower (at least ten times) than that required for conventional MRI, making this technology relatively inexpensive (as compared to MRI).

Conventional NMR base MRI imaging can also be performed. In this case, the superparamagnetic particles serve as the relaxation T₂ contrast agent. Standard MRI equipment can be used here.

Since the nanoparticles are superparamagnetic, they do not exert magnetic force to each other and form clusters at zero magnetic field (Standley and Vaughan (1969) Electron Spin Relaxatin Phenomena in Solids, Plenum Press). This makes sample preparation and particle delivery very simple. In certain embodiments, the superparamagnetic nanoparticles can be coated with certain inorganic or organic targeting moieties (e.g. lectins, antibodies, nucleic acids, chelates, etc.) in order for them to be delivered and attached to the targeted diseased cells, viruses or pathogens. In certain embodiments, targeting moieties are coupled to an epitope tag or chelate. The targeting moiety is administered to the cell, tissue, organ, or organism whereby it localizes at the desired target. Then the superparamagnetic nanoparticles attached to the corresponding binding moiety for the epitope tag is administered. The nanoparticles associate with the bound targeting moietie(s) thereby specifically/preferentially localizing the nanoparticles at the target site(s).

By applying, e.g., pulsed RF radiation power, the nanoparticles at the location that satisfies the equation (1) will be heated up to their Curie temperature. If the particle temperatures reach the Curie temperature, the particles lose their magnetic correlation and become paramagnetic. The spin population difference is then dramatically reduced and, as a result, the absorption power will go down. This effect gives the nanoparticles a convenient self-regulating mechanism to prevent over heating. Materials with a proper spin relaxation time constant (Poole (1983) Electron Spin Resonance (2nd Edition), A Wiley-Interscience Pub.) and Curie temperature can be chosen to form the nanoparticles to achieve optimized heating and therapeutic effects. Different sized nanoparticles can also be chosen to achieve the best delivery effect.

The present invention exploits existing biomedical and MRI technologies. For example, similar particles (e.g., oxides) have been used extensively as contrast agents in MRI applications. The linking chemistry for them has been well developed with many commercial kits available. The existing MRI technologies and equipments can be readily borrowed for this technology.

I. Calculation of the Resonance Heating Effect with Superparamagnetic Particles

Due to the spin-lattice interaction of superparamagnetic particles, the absorbed microwave energy by spin resonance will be converted to thermal energy after precessing electron spins are relaxed. A simple calculation can be performed with YIG (Y₃Fe₅O₁₂) nanopowder. YIG is a ferrimagnetic material having a net magnetization of 1400 emu/cm³ at room temperature (Goldman (1990) Modern Ferrite Technology, Van Nostrand Reinhold), or 1.5×10¹⁰ spins/μm³. Assuming the microwave frequency is 200 MHz, the relaxation time T₁ is 1 μs (Goldman (1990) Modern Ferrite Technology, Van Nostrand Reinhold; LeCraw and Spencer (1967) J. Phys. Soc. Jap. 17(Supplement B-I): 401), the microwave absorption power P for single spin is given by:

$$P = \frac{hf}{{T}_{2}} = 1.33 \times {10}^{- 19} W \cdot {spin}^{- 1}$$

The power absorbed per unit volume is:
Pᵥₒₗᵤₘₑ=P×1.5×10¹⁰=2.0×10⁻⁹ W ·μm⁻³

If this energy is used to heat up the surrounding water with volume 10 times that of the YIG particle, using thermal capacitance of water of 4.2 Jcm⁻³C°⁻¹ and adiabatic conditions, the heating rate of the YIG-water region is given by:
R_{T}=Pᵥₒₗᵤₘₑ/4.2×10⁻¹² Jμm⁻³C°⁻¹×10=47.6C°s⁻¹

This heating rate is rapid enough to kill nearby cells. We believe above assumptions are conservative and that more realistic conditions give rise to more effective heating of the target(s).

II. Calculation of Input Microwave Power.

We have also performed calculations on the saturation power necessary to excite all spins of the nanoparticles in a selected area.

To activate the spin resonance of ferromagnetic particles distributed in a large volume of human body, a relatively intense power is necessary. If we use a coil as microwave radiator, and a one-dimensional gradient magnetic field is used to realize the computed tomography, the effective volume is the product of the cross section area and the linear dimension of the ferromagnetic particles. Assuming the particle size is 10 nm, the necessary power per unit area is
Pₐᵣₑₐ=Pᵥₒₗᵤₘₑ•10 nm=20 W·m⁻²
Here we simply assume that all the localized microwave power will be absorbed by the ferromagnetic particles to activate the spin resonance. Depending on the coil impendence, the input power to the coil can be calculated with the required Pₐᵣₑₐ. This level of power is very simple to realize in practical applications.
III. Instrument Set-Up.

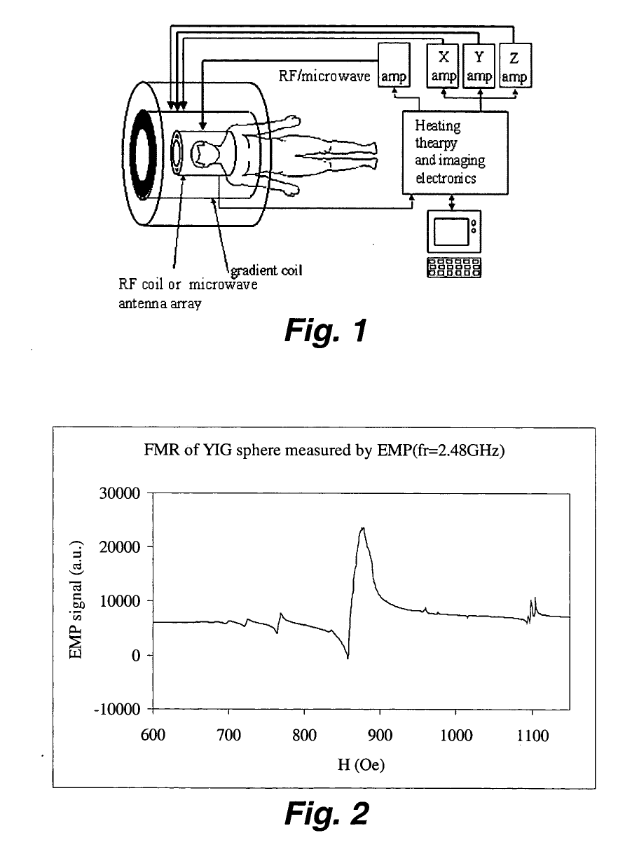

FIG. 1 illustrates a instrument set-up used for characterization of particle spin resonance detection and heating therapy. The setup is similar to the conventional MRI setup with the heating component integrated into it. Driven by the control electronics through X, Y, Z amplifier, the gradient coil can provide gradient magnetic field variable in 3 dimension which is necessary to localized the specific region of the tested sample or human body. The RF coil or alternatively the microwave antenna array is used as heating and spin resonance detection element. The 3D spin resonance imaging can be taken first with small microwave/RF power to locate the area where the heat therapy is necessary. Then the gradient field can be applied so that only the section contains the interesting region can satisfy the spin resonance condition. The heat therapy is processed then by adding higher power microwave through the RF coil to the whole body or microwave antenna array to a more focused region.

A typical ferromagnetic resonance of YIG sphere (diameter 0.3 mm) is shown in FIG. 2. The line width of the resonant peak shown in the resonance width is about 30 Oe, which is close to the reported value for YIG ceramic.

IV. Spin Resonance Line Width and Heating Effect of Different Materials.

A preliminary calculation outlining the heating capabilities of the YIG sphere was provided above. In certain embodiments, however, it is desirable to optimize several parameters: The relationship between the spin resonance line width and the power absorption rate and the heating efficiency; the particle size and its effect on the power absorption rate and the heating efficiency; and the best operating frequency.

The spin resonance line width is inversely proportional to the lifetime of the spin energy level. The broader the line width, the shorter the life time, which means the material may convert microwave energy into thermal energy more quickly. Therefore, higher levels of saturation power can be achieved. Broader line width materials, however, will decrease the microwave absorption efficiency when the RF source has a narrow bandwidth. For the purpose of selective excitation of the magnetic resonation and high spatial resolution in both treatment and imaging, the frequency bandwidth is desirably narrow. Therefore, optimized line width(s) should exist for the purpose of heating therapy and simultaneous imaging.

The line width of the spin resonance can readily be detected using simple modifications to the set up shown in FIG. 1. To avoid absorption by water or biological fluids in the microwave region, the microwave frequency should be as low as possible since water absorption increase with the microwave frequency. On the other hand the heating rate of magnetic resonance drops at lower frequency. In certain embodiments, the optimized frequency should be in the range of about 50 to about 2000 MHz, preferably about 100 to about 1000 MHz, more preferably from about 500 to about 1000 MHz. In this frequency range an RF coil can be used as an RF transmitter and receiver. Compared to the resonator detector shown in FIG. 1, the RF coil may have a higher RF power transfer efficiency and can achieve uniform RF distribution in relatively larger regions. It also provides an open environment that is convenient to characterize the heating efficiency. Phase array antenna can be used here for radiation and detection of RF wave.

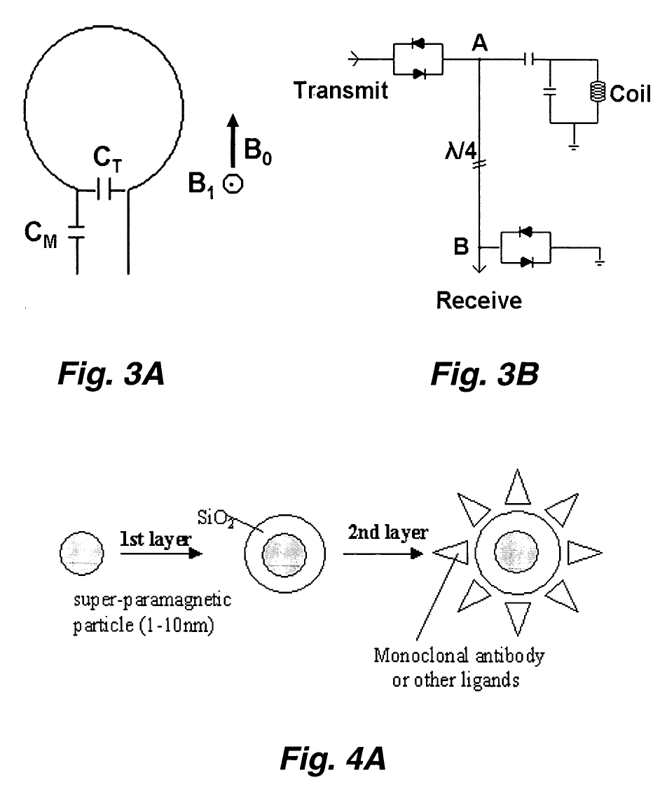

A surface coil can be applied to small volume for sample detection. In its simplest form it is a coil of wire coupled with a capacitor in parallel. The inductance of the coil, and the capacitance form a resonant circuit, which is tuned to have the same resonant frequency as the spins to be detected. A second capacitor can be added in series with the coil, as shown in FIG. 3A, to match the coil impedance to, e.g., 50Ω. To prevent excitation pulse saturation or break-down of the receiver electronics which are designed to detect signals up to 6 orders lower than the input power, a simple protection circuit can be used as shown in FIG. 3B. To achieve a better signal noise ratio, the pulse RF signal can be used to replace the CW microwave signal. T his can be realized with the same microwave synthesizer by simply adding the pulse modulation control.

In certain embodiments, to study and characterize the heating effect of the particles, two methods can be used to measure the temperature increase cause by the spin resonance. In the first method, an infrared thermometer, e.g., with a temperature sensitivity of 1° C. can be used to monitor the radiation from the heated sphere (nanoparticles). Since it is impossible to focus the detection area as small as a nanoparticles due to the Abbe diffraction limit, a cluster of such powder, e.g., in a small glass tube can be used for the detection. In a second approach, a temperature sensitive paint (TSP) can be used to coat the nanoparticles. For example, a diluted layer of nanoparticles can be coated on a piece of glass slide. Then a thin layer of TSP can be coated. The heating effect is observed by the color change of the TSP. The temperature sensitivity of this method may be lower than the infrared thermometer, but it could directly monitor the surface temperature change of individual nanoparticles. When the sphere size smaller than 300 nm, the temperature change of individual nanoparticles will not able to be detected by this method. One can only estimate the temperature by the overall color change of the TSP coating covered on a cluster of spheres.

The relationship between heating up efficiency and nanopariclesize can also be empiracly determined and optimized. Ideally, the nanoparticles size will not affect the heating efficiency if the heat generated by the RF absorption is only used to heat up the same volume. It is noted that there are several companies that produce commercially available superparamagnetic (e.g. ferrimagnetic, ferromagnetic, etc.) nanoparticles (e.g., Deltronic Inc). In certain embodiments, the nanoparticles range in size from about 1 nm to about 10 μm, preferably from about 10 nm to about 1 μm, more preferably from about 10 nm to about 100 nm.

Ferromagnetic resonance (FMR) is the electron spin resonance (ESR) in ferromagnetic or ferrimagnetic media. Due to long-range order of electron spins in ferro- or ferri-magnetic materials, the spin population difference is nearly one at room temperature. As a consequence, the sensitivity of FMR will not be reduced by the Boltzmann factor at room temperature for spin population difference, even if the radiation frequency is dramatically reduced. Therefore, this approach permits the use of radio frequency high FMR signals for heating and/or imaging in biological organisms and also provides high heating efficiency. In certain embodiments, ferrimagnetic materials with narrow resonance line width are used.

The importance of narrow resonance line width for high near-resonance sensitivity is seen in both the real and imaginary parts of the complex permeability μ=μ′+iμ″. In microwave (RF) circuits, μ′ controls the signal phase and μ″ controls the energy absorption or circuit Q factor. Their relations as a function of angular frequency ω can be expressed as:

$$\begin{matrix}\begin{matrix}{μ}^{′} = 1 + \frac{γ 4 π M ( ω - {ω}_{0} )}{( {ω}^{2} - {ω}_{0}^{2} ) + {{γ}^{2} ( Δ H )}^{2}} \\ \approx 1 + \frac{γ 4 π M ( ω - {ω}_{0} )}{{{γ}^{2} ( Δ H )}^{2}} ( near resonance )\end{matrix} \\ and & 3 A \\ \begin{matrix}{μ}^{″} = \frac{γ 4 π Mγ ( Δ H )}{( {ω}^{2} - {ω}_{0}^{2} ) + {{γ}^{2} ( Δ H )}^{2}} \\ \approx \frac{4 π M}{Δ H} ( at resonance )\end{matrix} , & 3 B\end{matrix}$$

where 4πM is the magnetization comprising the volume density of individual magnetic moments m, ω₀ is the resonance frequency, and ΔH is the line width. The factor γ is the gyromagnetic constant and is derived from the Larmor precession relation between frequency and field, given by:

$${ω}_{0} = γ H = \frac{g \vert e \vert}{2 mc} H , \qquad ( 4 )$$

where e is the electron or proton charge, m is the particle mass and c is the velocity of light, and g (˜2 for spins) is the spectroscopic splitting factor. Note that e is the same magnitude for both protons and electrons, but mₙ for protons is greater than mₑ for electrons by a factor 1836, thereby reducing the resonance frequency by a factor of more than 10³ for a given magnetic field intensity H.

From equation 3B, the imaginary part of susceptibility μ″ is proportional to 1/ΔH (ΔH is line width), and μ″ is directly related to the RF energy absorption of the material, which means that materials with narrow spin resonance line width will have high RF absorption efficiency and can be easily heated for a given single excitation frequency coincide with the spin resonance frequency.

In certain embodiments, the RF will range from about 400 MHz to about 1 GHz to heat the material. In this context, a typical/reasonable pulse width is about 1 μs, which corresponds to a line width of 1 MHz and quality factor of about 500˜1000. If the line width of selected material is too broad (low quality factor), the absorption band of the material will not be covered effectively by the RF pulse spectrum, which will also decrease the heating efficiency. Thus the spin resonance quality factor of the selected material should be larger than 10, more preferably larger than about 50, still more preferably larger than about 100, 200, or 500. In certain embodiments, the spin resonance quality factor (Q) ranges from these values up to about 800, 1000, 15000, 2000, or 3000. In certain embodiments, the Q factor ranges from about 100 to about 1000.

Several factors contribute to the line width, chief among which are (1) spin-lattice interactions of individual spins, characterized by a relaxation time τ₁, and (2) incoherent precession phasing of spins, characterized by a relaxation time τ₂ that arises from misaligned spins coupled by dipolar interactions. Precession phase decoherence can also occur in exchange ordered electron spin systems by spin wave generation, particularly in higher power cases where crystal imperfections or non-uniform RF fields exist in a specimen having dimensions greater than the wavelength of the RF signal. These mechanisms are generally considered to be homogeneous and produce a Lorentzian line shape.

Inhomogeneities can cause severe broadening by creating local regions of different resonance frequencies in a Gaussian-type distribution. Most common among these cases are polycrystalline ferromagnetic specimens with crystal grains of random crystallographic orientation with varying magnetic anisotropy bias fields and structural inhomogeneities such as nonmagnetic phases, porosity and grain boundaries that can broaden the effective ΔH of a typical ferrite by more than a hundred oersteds. In small specimens with rough surfaces, demagnetization effects on line width, similar to those of bulk porosity, have been observed. For this reason, the discussion of FMR that follows focuses primarily on relatively polished single crystal specimens where only the homogeneous broadening effects from the relaxation rates τ₁⁻¹ and τ₂⁻¹.

For homogeneous relaxation broadening,
ΔH=(γτ)⁻¹ (5)

where the relaxation time τ can be a resultant of both τ₁ and τ₂ contributions, but is generally dominated by only one of them. Relaxation rates of paramagnetic systems are influenced primarily by τ₂⁻¹, with the possible exception of certain electron cases where fast relaxing ions allow two-phonon Raman processes to render τ₁⁻¹ large enough to approach or exceed τ₂⁻¹. With ferromagnetic specimens, the spin-spin relaxation rate in ideal situations is effectively zero because of complete spin alignment means perfect precession phase coherence. Although τ₁⁻¹ becomes the dominant relaxation parameter, only selected ions can fulfill the goal of narrow line width. Estimated values of these parameters are listed in Table 1 for typical situations.

TABLE 1
Estimates of gyromagnetic resonance parameters at T = 300° K.

| | 4πM | τ₁ | τ₂ | ΔH | μ″ |
|---|---|---|---|---|---|
| | (G) | (sec) | (sec) | (Oe) | — |
| NMR | ˜2 (conc.) | >10⁴ | ˜10⁻⁴ | ˜0.05 | ˜40 |
| EPR | 20 (dilute) | >10⁻⁶ | ˜10⁻⁷ | ˜5 | ˜4 |
| (Fe³⁺) | 2000 (conc.) | | ˜10⁻⁹ | ˜500 | ˜4 |
| FMR | 2000 (conc.) | >10⁻⁶ | → ∞ | <0.5 | 4000 |
| (Fe³⁺) | | | | | |

The resonance frequency ω₀ can vary with orientation of the specimen in different ways. For paramagnets, γ can be sensitive to crystallographic direction, and in some case, range widely. However, γ is relatively isotropic in ferrimagnets with d⁵ or d⁷ magnetic ions. The main sources of anisotropy come from surface poles that induce demagnetizing fields proportional to 4πμM inside the specimen, and from fields proportional to ratio of the magnetocrystalline anisotropy fields that are associated with specific crystallographic axes.

For a fully magnetized ellipsoidal specimen with H and M aligned with the z-axis, the resonance frequency is expressed as
ω₀=γHᵢ, 6
where the internal field for resonance is given by

$${H}_{r} = {\{ \begin{matrix}[ H + ( {H}_{Kx} - {H}_{Kz} ) + ( {N}_{Dx} - {N}_{Dz} ) 4 π M ] \times \\ [ H + ( {H}_{Ky} - {H}_{Kz} ) + ( {N}_{Dy} - {N}_{Dz} ) 4 π M ]\end{matrix} \}}^{\frac{1}{2}} \qquad 7$$

The subscripts x and y refer to the two axes of the ellipsoid that are orthogonal to the z direction of H in the coordinate system selected. Note that Hᵢ reduces to the applied field H when all of the demagnetizing factors are zero.

For resonance to occur with H along the z-axis, H_{rf} must have a component in the xy-plane, but values of the H_{K} anisotropy fields and the N_{D} factors will be sensitive to the direction of H within the plane. Applied to the limiting case of a thin flat plate with N_{Dx}, =1, and N_{Dy}, N_{Dz}=0, and H_{K} terms ignored, equation 6 simplifies to:

$${ω}_{0} = {γ [ H ( H + 4 π M ) ]}^{\frac{1}{2}} ( H in plane ) \\ {ω}_{0} = γ ( H - 4 π M ) ( H normal to plane ) \qquad 8$$

For a long slender cylinder (acicular particle) aligned with the z-axis, N_{Dx}, N_{Dy=}½, and N_{Dz}=0. The resonance frequency is then:

$${ω}_{0} = γ ( H + 2 π M ) ( H parallel to long axis ) \\ {ω}_{0} = {γ [ H ( H - 2 π M ) ]}^{\frac{1}{2}} ( H normal to long axis ) \qquad 9$$

For a sphere, N_{Dx}=N_{Dy}=N_{Dz=}⅓, and the shape demagnetizing factors cancel, so that Hᵢ=H.
ω₀=γH 10

As a consequence, care is taken in selecting specimen shapes. From equation 10, it is clear that spherical particles are most suitable for this purpose. In addition, dispersal of the individual ferrimagnets is also important to avoid dipolar interactions on a macroscopic scale, e.g., super-paramagnets.

V. The Instrument Design of the Gradient Magnetic Field for Imaging Assisted, Differential Heating Therapy for Cancer.

For 3D heating capabilities with larger specimens (organisms), the surface coil is preferably replaced with a commercial available birdcage coil, which can provide uniform RF distribution in bigger volume. To realize localized heating and spatially resolved imaging, a magnetic field gradient is provided. FIGS. 6A and 6B illustrate the generation of gradient field for 3-D heating and/or imaging.

Magnetic field gradients are spatially dependent variations in the magnetic field created by electrical DC currents in specifically designed coil arrangements. For example, a linear magnetic field gradient that varies spatially along the z direction of the main magnet can be produced using a Maxwell pair of coils as pictured in FIG. 6B. Such a magnetic field, when applied to a sample of homogeneous material like water, causes the spins on one side of the sample with respect to the z direction to have a different frequency from spins on the other side of the sample. A distribution of frequencies will be obtained along the sample. The amount of magnetization at each frequency will be the integral of the signal along a surface perpendicular to the applied field gradient. An x gradient is obtained using a coil configuration as shown in FIG. 6A, and need only be rotated by 90 degrees to obtaiun any gradient. Both of these make fields that add or subtract from the main magnetic field pointing along z but the magnetic field strength varies in the x or y direction.

The 3D heating and imaging setup preferably controls the gradient field and RF pulse in a specific time sequence. Software controlling the device can offer the following functions: 1) Control of the gradient field to realize the planar selection for heating and magnetic resonance detection; 2) Control of the RF pulse sequence according to the applications. In certain embodiments, for heating, a continuous 180° pulse is provided with period related to the relaxation time of the magnetic resonance. For imaging, In certain embodiments, a 90° pulse is provided to observe the relaxation signal. 3) The FFT functions can be used to analyze the line width of the spin resonance (Ernst et al. (1987) Principles of Nuclear Magnetic Resonance in One and Two Dimensions, Clarendon Press Oxford) and reconstruct the image when phase encoding and frequency encoding pulse is used to realize the magnetic resonance imaging.

VI. Superparamagnetic Material Selection.

Compared to NMR, electron paramagnet resonance offers larger individual magnetic moments, but has broader associated line widths resulting from relaxation times that are shortened by spin-orbit coupling in all cases except the half-filled d shell ions, i.e., 3d⁵ of Fe 3+, Mn²⁺ or rare earth 4f⁷ of Gd 3+, Eu²⁺. Strong dipolar coupling also reduces τ₂ when concentrations of paramagnetic centers are increased in attempts to raise the dc susceptibility.

For selection of particles, single-crystal ferrimagnetic spheres offer the advantages of high detectability through large magnetizations and narrow FMR lines. For example, yttrium-iron garnet Y₃Fe₅O₁₂ and γ-Fe₂O₃ are two well-known materials suitable for this application. Different dopants can be added to lower the spin resonance frequencies of these materials for medical applications. Magnetic garnets and spinels are also chemically inert and indestructible under normal environmental conditions.

An illustrative list of potential dilutant ions for the generic {c}₃(a)₂[d]₃O₁₂ and spinel A[B]₂O₄ ferrite compounds that preserve produce different ω₀ values while preserving the narrow ΔH requirement is presented in Table 2. Among those ions to be preferably avoided are those with fast spin-lattice relaxation rates, specifically members of the 3d or 4f transition series without half-filled shells, particularly Co²⁺, Fe²⁺, or any of the lanthanide (rare earth) series not listed in Table 2.

TABLE 2
Ferrite dilution ions for preserving narrow line width.

| Garnet | | Spinel | |
|---|---|---|---|
| {c}₃(a)₂[d]₃O₁₂ | | A[B]₂O₄ | |
| {c} | (a) octahedral | A | [B] |
| dodecahedral | [d] tetrahedral | tetrahedral | octahedral |
| Y³⁺ (highest purity) | Fe³⁺ | Fe³⁺ | Fe³⁺ |
| La³⁺ (highest purity) | Mn²⁺ | Mn²⁺ | Mn²⁺ |
| Gd³⁺ (highest purity) | Ru³⁺ | Ru³⁺ | Ru³⁺ |
| Eu²⁺ (highest purity) | Cu¹⁺ | Cu¹⁺ | Cu¹⁺ |
| Na¹⁺ | V³⁺ [d], Ni²⁺ (a) | V³⁺ | Ni²⁺ |
| K¹⁺ | Cr⁴⁺ [d], Cu³⁺ (a) | Cr⁴⁺ | Cu³⁺ |
| Rb¹⁺ | Mo⁴⁺ [d], Cr³⁺ (a) | Mo⁴⁺ | Cr³⁺ |
| Tl¹⁺ | W⁴⁺ [d], Mo³⁺ (a) | W⁴⁺ | Mo³⁺ |
| Ag¹⁺ | Nb³⁺ [d], W³⁺ (a) | Nb³⁺ | W³⁺ |
| Au¹⁺ | Zn²⁺ | Zn²⁺ | Zn²⁺ |
| Hg¹⁺ | Mg²⁺ | Mg²⁺ | Mg²⁺ |
| Ca²⁺ | Al³⁺ | Al³⁺ | Al³⁺ |
| Sr²⁺ | Ga³⁺ | Ga³⁺ | Ga³⁺ |
| Ba²⁺ | In³⁺ | In³⁺ | In³⁺ |
| Hg²⁺ | Sc³⁺ | Sc³⁺ | Sc³⁺ |
| Pb²⁺ | Ti⁴⁺ | Ti⁴⁺ | Ti⁴⁺ |
| Bi³⁺ | Zr⁴⁺ | Zr⁴⁺ | Zr⁴⁺ |
| In³⁺ | Hf⁴⁺ | Hf⁴⁺ | Hf⁴⁺ |
| Sc³⁺ | Si⁴⁺ | Si⁴⁺ | Si⁴⁺ |
| | Ge⁴⁺ | Ge⁴⁺ | Ge⁴⁺ |
| | Sn⁴⁺ | Sn⁴⁺ | Sn⁴⁺ |
| | V⁵⁺ | V⁵⁺ | V⁵⁺ |
| | Nb⁵⁺ | Nb⁵⁺ | Nb⁵⁺ |
| | Ta⁵⁺ | Ta⁵⁺ | Ta⁵⁺ |
| | P⁵⁺ | P⁵⁺ | P⁵⁺ |
| | As⁵⁺ | As⁵⁺ | As⁵⁺ |
| | Sb⁵⁺ | Sb⁵⁺ | Sb⁵⁺ |

VII. Targeting Moieties.

In certain embodiments, the superparmagnetic nanoparticles described herein are coupled to one or more targeting moieties so that they specifically or preferentially bind to certain target(s) (e.g. cancer cells). Generally speaking, materials which can be employed as targeting ligands include, but are not limited to, proteins, including antibodies, glycoproteins and lectins, peptides, polypeptides, saccharides, including mono- and polysaccharides, vitamins, steroids, steroid analogs, hormones, cofactors, bioactive agents, and genetic material, including nucleosides, nucleotides and polynucleotides.

The targeting moieties which may be incorporated in the compositions of the present invention are preferably substances that are capable of targeting (e.g. specifically or preferentially binding) receptors, and/or particular cell-surface markers, and/or particular cells, and/or particular organs or tissues in vivo.

With respect to the targeting of cancers (e.g., solid tumors or cancer cells), it is noted that a number of cancer-specific markers are known to those of skill in the art. Such markers include, but are not limited to C-myc, p53, Ki67, erbB-2, Her2, Her4, BRCA1, BRCA2, Lewis Y, CA 15-3, G250, HLA-DR cell surface antigen, CEA, CD2, CD3, CD7, CD19, CD20, CD22, integrin, EGFr, AR, PSA, carcinoembryonic antigen (CEA), the L6 cell surface antigen (see, e.g., Tuscano et al. (2003) Neoplasia, 3641-3647; Howell et al. (1995) Int J Biol Markers 10: 126-135; Marken et al. (1992) Proc. Natl. Acad. Sci. U.S.A. 89: 3503-3507, 1992), growth factor receptors, and/or various intracellular targets (e.g. receptors, nucleic acids, phosphokinases, etc.) and the like.

In certain embodiments, targeting moieties can be selected for targeting antigens associated with breast cancer, such as epidermal growth factor receptor (EGFR), fibroblast growth factor receptor, erbB2/HER-2 and tumor associated carbohydrate antigens (Siegall (1994) Cancer, 74(3): 1006-12). CTA 16.88, homologous to cytokeratins 8, 18 and 19, is expressed by most epithelial-derived tumors, including carcinomas of the colon, pancreas, breast, ovary and lung. Thus, antibodies directed to these cytokeratins, such as 16.88 (IgM) and 88BV59 (IgG3k), which recognize different epitopes on CTA 16.88 (Jager et al. (1993) Semin. Nucl. Med., 23(2): 165-79), may be employed as targeting ligands. For targeting colon cancer, anti-CEA antibodies, e.g., IgG Fab′ fragments may be employed as targeting ligands. In certain embodiments, chemically conjugated bispecific anti-cell surface antigen, anti-hapten Fab′-Fab antibodies may also be used as targeting moieties. The MG series monoclonal antibodies may be selected for targeting, for example, gastric cancer.

There are a variety of cell surface epitopes on epithelial cells for which targeting ligands may be selected. For example, the protein human papilloma virus (HPV) has been associated with benign and malignant epithelial proliferations in skin and mucosa. Two HPV oncogenc proteins, E6 and E7, may be targeted as these may be expressed in certain epithelial derived cancers, such as cervical carcinoma (see, e.g., (1994) Curr. Opin. Immunol. 6(5: 746-754). Membrane receptors for peptide growth factors (PGF-R), which are involved in cancer cell proliferation, cam also be selected as tumor antigens (see, e.g, (1994) Anticancer Drugs, 5(4): 379-393). Also, epidermal growth factor (EGF) and interleukin-2 may be targeted with suitable targeting ligands, including peptides, which bind these receptors. Certain melanoma associated antigens (MAA), such as epidermal growth factor receptor (EGFR) and adhesion molecules ((1994) Tumor Biol., Vol. 15(4): 188-202), which are expressed by malignant melanoma cells, can be targeted with the compositions provided herein. The tumor associated antigen FAB-72 on the surface of carcinoma cells can may also be selected as a target. These targets are intended to be illustrative and not limiting.

In certain embodiments, an example of a protein which may be preferred for use as a targeting ligand is Protein A, which is protein that is produced by most strains of Staphylococcus aureus. Protein A is commercially available, for example, from Sigma Chemical Co. (St. Louis, Mo.). Protein A may then be used for binding/targeting a variety of IgG antibodies. Generally speaking, peptides which are particularly useful as targeting ligands include natural, modified natural, or synthetic peptides that incorporate additional modes of resistance to degradation by vascularly circulating esterases, amidases, or peptidases. One very useful method of stabilization of peptide moieties incorporates the use of cyclization techniques. As an example, the end-to-end cyclization whereby the carboxy terminus is covalently linked to the amine terminus via an amide bond can be useful to inhibit peptide degradation and increase circulating half-life. Additionally, a side chain-to-side chain cyclization is also particularly useful in inducing stability. In addition, an end-to-side chain cyclization can be a useful modification as well. In addition, the substitution of an L-amino acid for a D-amino acid in a strategic region of the peptide may offer resistance to biological degradation. Suitable targeting ligands, and methods for their preparation, will be readily apparent to one skilled in the art, once armed with the disclosure herein.

VIII. Attaching the Targeting Moiety to the Superparamagnetic Particle.

In one embodiment, the targeting molecule (e.g., a HER2 antibody, an anti Le^{Y} antibody, etc.) is chemically conjugated to the superparamagnetic nanoparticle. Means of chemically conjugating molecules are well known to those of skill. In certain embodiments, multiple targeting moieties are joined to each nanoparticles, In certain embodiments, multiple nanoparticles are attached to each targeting moiety, and in other embodiments, one targeting moiety is attached to each nanoparticles. The attachment can be direct or through a linker.

The procedure for attaching an nanoparticles to an antibody or other targeting moiety will vary according to the chemical structure of the targeting moiety. Polypeptides, for example, typically contain variety of functional groups; e.g., carboxylic acid (COOH) or free amine (—NH₂) groups, that are available for reaction with a suitable functional group on, e.g. a derivatized superparamagnetic nanopartilcle to bind the targeting moiety thereto.

In certain embodiments, the targeting moiety and/or the nanoparticles(s) can be derivatized to expose or attach additional reactive functional groups. The derivatization may involve attachment of any of a number of linker molecules such as silanes, crosslinking reagents such as gluteraldehyde, and the like. Such reagents are available from Pierce Chemical Company, Rockford Ill.

A “linker”, as used herein, is a molecule that can be used to join the targeting moiety to the superparamagnetic nanoparticle. The linker is capable of forming covalent bonds to both the targeting moiety and to the typically derivatized nanoparticle. Suitable linkers are well known to those of skill in the art and include, but are not limited to, straight or branched-chain carbon linkers, heterocyclic carbon linkers, peptide linkers, and the like. Where the targeting moiety comprises a polypeptides, the linker can be joined to the constituent amino acids through their side groups (e.g., through a disulfide linkage to cysteine) or to the alpha carbon amino and carboxyl groups of the terminal amino acids.

A bifunctional linker having one functional group reactive with a group on a nanoparticle, and another group reactive with, for example, an antibody, may be used to form the desired immunoconjugate. Alternatively, derivatization may involve chemical treatment of the targeting moiety, e.g., glycol cleavage of the sugar moiety of a glycoprotein antibody with periodate to generate free aldehyde groups. The free aldehyde groups on the antibody may be reacted with free amine or hydrazine groups on a linker or nanoparticles to bind the nanoparticles thereto (see, e.g., U.S. Pat. No. 4,671,958). Procedures for generation of free sulfhydryl groups on polypeptides, such as antibodies or antibody fragments, are also known (see, e.g., U.S. Pat. No. 4,659,839).

Many procedure and linker molecules for attachment of various compounds including radionuclide metal chelates, toxins and drugs to proteins such as antibodies are known (see, e.g., European Patent Application No. 188,256; U.S. Pat. Nos. 4,671,958, 4,659,839, 4,414,148, 4,699,784; 4,680,338; 4,569,789; and 4,589,071; and Borlinghaus et al. (1987) Cancer Res. 47: 4071-4075). In particular, production of various iimmunoconjugates is well-known within the art and can be found, for example in “Monoclonal Antibody-Toxin Conjugates: Aiming the Magic Bullet,” Thorpe et al., Monoclonal Antibodies in Clinical Medicine, Academic Press, pp. 168-190 (1982), Waldmann (1991) Science, 252: 1657, U.S. Pat. Nos. 4,545,985 and 4,894,443.

In some circumstances, it is desirable to free the superparamagnetic nanoparticles from the targeting moiety when conjugate has reached its target site. Therefore, targeting moiety/nanoparticles conjugates comprising linkages that are cleavable in the vicinity of the target site can be used when the nanoparticles is to be released at the target site. Cleaving of the linkage to release the agent from the targeting moiety can be prompted by enzymatic activity or conditions to which the conjugate is subjected either inside the target cell or in the vicinity of the target site. When the target site is a tumor, a linker which is cleavable under conditions present at the tumor site (e.g. when exposed to tumor-associated enzymes or acidic pH) may be used.

A number of different cleavable linkers are known to those of skill in the art. See U.S. Pat. Nos. 4,618,492; 4,542,225, and 4,625,014. The mechanisms for release of an agent from these linker groups include, for example, irradiation of a photolabile bond and acid-catalyzed hydrolysis. U.S. Pat. No. 4,671,958, for example, includes a description of immunoconjugates comprising linkers which are cleaved at the target site in vivo by the proteolytic enzymes of the patient's complement system. In view of the large number of methods that have been reported for attaching a variety of radiodiagnostic compounds, radiotherapeutic compounds, drugs, toxins, and other agents to antibodies and other targeting moieties, one skilled in the art will be able to determine a suitable method for attaching a given targeting moiety to the nanoparticles(s) of interest.

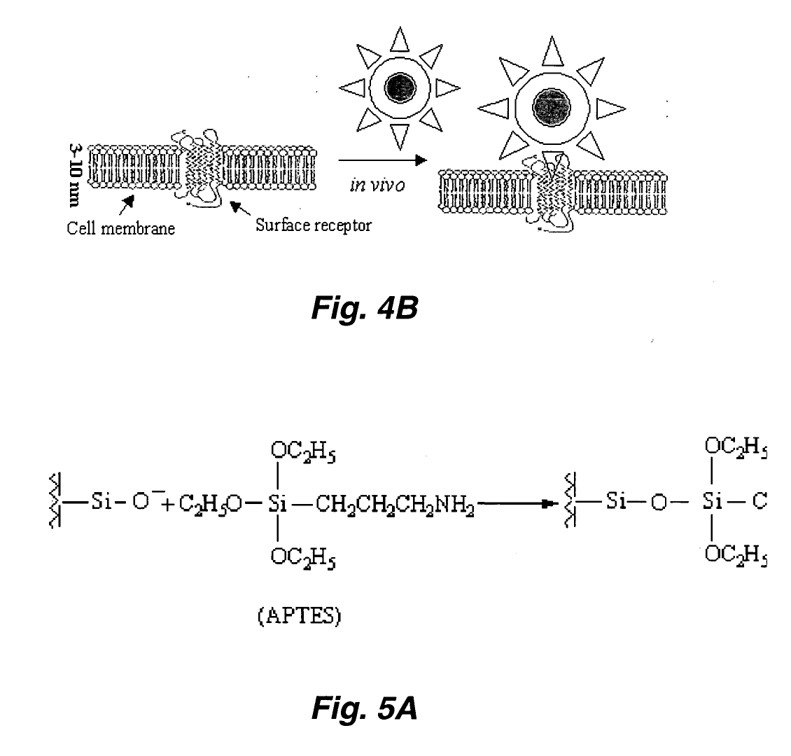

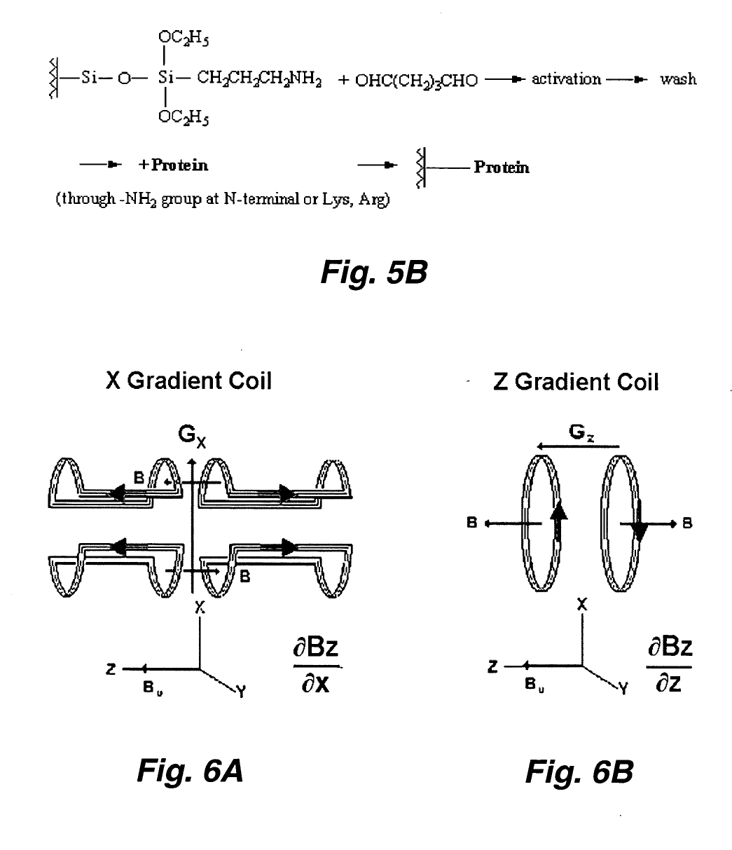

In certain particularly preferred embodiments the nanoparticles are attached to targeting moieties (e.g., to antibodies or other high-affinity ligands) by coating/derivatizign the particles with with a layer of organic molecules or inorganic molecules (e.g., SiO₂) to produce a layer ranging, e.g., from about 1 nm to about 100 nm, preferably from about 10 nm to about 50 nm in thickness (see, e.g., FIG. 4A). The surface of the layer can then, optionally be derivatized (e.g. silane, gluteraldehyde, etc.) and targeting moieties can then can then be attached to the modified particle surface for the cell surface recognition (FIG. 4B) and cell imaging purpose with MRI. It is noted thata silanization of SiO₂ surface by, e.g., 3-aminopropyl-triethoxysilane (APTES) can result in an active amine group (FIG. 5A). Therefore proteins and ligands with free carboxyl group can be readily attached directly (FIG. 5B).

In certain preferred embodiments, the nanoparticles are joined to an antibody or to an epitope tag through a chelate. The targeting moiety bears a corresponding epitope tag or antibody so that simple contacting of the targeting moiety to the nanoparticles results in attachment of the targeting moiety with the nanoparticle. The combining step can be performed before the targeting moiety is used (targeting strategy) or the target tissue can be bound to the targeting moiety before the nanoparticles chelate is delivered. Methods of producing chelates suitable for coupling to various targeting moieties are well known to those of skill in the art (see, e.g., U.S. Pat. Nos. 6,190,923, 6,187,285, 6,183,721, 6,177,562, 6,159,445, 6,153,775, 6,149,890, 6,143,276, 6,143,274, 6,139,819, 6,132,764, 6,123,923, 6,123,921, 6,120,768, 6,120,751, 6,117,412, 6,106,866, 6,096,290, 6,093,382, 6,090,800, 6,090,408, 6,088,613, 6,077,499, 6,075,010, 6,071,494, 6,071,490, 6,060,040, 6,056,939, 6,051,207, 6,048,979, 6,045,821, 6,045,775, 6,030,840, 6,028,066, 6,022,966, 6,022,523, 6,022,522, 6,017,522, 6,015,897, 6,010,682, 6,010,681, 6,004,533, and 6,001,329).

IX. Pharmaceutical Compositions.

The superparamagnetic nanoparticles or nanoparticles attached to targeting moieties of this invention (particularly those specific for cancer or other pathologic cells) can be useful for parenteral, topical, oral, or local administration (e.g. injected into a tumor site), aerosol administration, or transdermal administration, for prophylactic, but principally for therapeutic treatment. The pharmaceutical compositions can be administered in a variety of unit dosage forms depending upon the method of administration. For example, unit dosage forms suitable for oral administration include powder, tablets, pills, capsules and lozenges. It is recognized pharmaceutical compositions of this invention, when administered orally, can be protected from digestion. This is typically accomplished either by complexing the active component (e.g. the targeting moiety) with a composition to render it resistant to acidic and enzymatic hydrolysis or by packaging the active ingredient(s) in an appropriately resistant carrier such as a liposome. Means of protecting components from digestion are well known in the art.

The pharmaceutical compositions of this invention are particularly useful for parenteral administration, such as intravenous administration or administration into a body cavity or lumen of an organ. The compositions for administration will commonly comprise a solution of the nanoparticles and/or nanoparticles attached to targeting moieties dissolved in a pharmaceutically acceptable carrier, preferably an aqueous carrier. A variety of aqueous carriers can be used, e.g., buffered saline and the like. These solutions are sterile and generally free of undesirable matter. These compositions can be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of chimeric molecule in these formulations can vary widely, and will be selected primarily based on fluid volumes, viscosities, body weight and the like in accordance with the particular mode of administration selected and the patient's needs.

The compositions containing the nanoparticles and/or nanoparticles attached to targeting moieties or a cocktail thereof (i.e., with other therapeutics) can be administered for therapeutic treatments. In therapeutic applications, compositions are administered to a patient suffering from a disease, e.g., a cancer, in an amount sufficient to cure or at least partially arrest the disease and its complications when appropriately utilized with electron spin resonance to effect heating of the nanoparticles. An amount adequate to accomplish this is defined as a “therapeutically effective dose.” Amounts effective for this use will depend upon the nature of the disease and the general state of the patient's health.

Single or multiple administrations of the compositions may be administered depending on the dosage and frequency as required and tolerated by the patient. In any event, the composition should provide a sufficient quantity of the compositions of this invention to effectively treat the patient.

It will be appreciated by one of skill in the art that there are some regions that are not heavily vascularized or that are protected by cells joined by tight junctions and/or active transport mechanisms which reduce or prevent the entry of macromolecules present in the blood stream.

One of skill in the art will appreciate that in these instances, the therapeutic compositions of this invention can be administered directly to the tumor site. Thus, for example, brain tumors can be treated by administering the therapeutic composition directly to the tumor site (e.g., through a surgically implanted catheter).

Alternatively, the therapeutic composition can be placed at the target site in a slow release formulation. Such formulations can include, for example, a biocompatible sponge or other inert or resorbable matrix material impregnated with the targeted nanoparticles, slow dissolving time release capsules or microcapsules, and the like.

Typically the catheter or time release formulation will be placed at the tumor site as part of a surgical procedure. Thus, for example, where major tumor mass is surgically removed, the perfusing catheter or time release formulation can be emplaced at the tumor site as an adjunct therapy. Of course, surgical removal of the tumor mass may be undesired, not required, or impossible, in which case, the delivery of the therapeutic compositions of this invention may comprise the primary therapeutic modality.

V. Kits.

In various embodiments, this invention nprovides kits for the practice of this invention. The kits can comprise one or more containers containing superparamagnetic nanoparticles as described herein. The nanoparticles can optionally be derivatized, e.g. for attachment to a targeting moiety. In certain embodiments, the nanoparticles are provided already attached to a targeting moiety. In certain embodiments, the nanoparticles and targeting moieties are provided separately and the kit further contains reagents for coupling targeting moieties to the nanoparticles. The kit is preferably designed so that the manipulations necessary to perform the desired reaction should be as simple as possible to enable the user to prepare from the kit the desired composition by using the facilities that are at his disposal. Therefore the invention also relates to a kit for preparing a composition according to this invention. In certain embodiments, the kit can optionally, additionally comprise a reducing agent and/or, if desired, a chelator, and/or instructions for use of the composition and/or a prescription for reacting the ingredients of the kit to form the desired product(s). If desired, the ingredients of the kit may be combined, provided they are compatible.

When kit constituent(s) are used as component(s) for pharmaceutical administration (e.g. as an injection liquid) they are preferably sterile and can, optionally be provided in a pharmacologically acceptable excipient. When the constituent(s) are provided in a dry state, the user should preferably use a sterile physiological saline solution as a solvent. If desired, the constituent(s) can be stabilized in the conventional manner with suitable stabilizers, for example, ascorbic acid, gentisic acid or salts of these acids, or they may comprise other auxiliary agents, for example, fillers, such as glucose, lactose, mannitol, and the like.

In certain embodiments, the kits additionally comprise instructional materials teaching the use of the compositions described herein (e.g., nanoparticles, derivatized nanoparticles, etc.) in electron spin resonance applications for selectively heating cells, tissue, organs, and the like.

While the instructional materials, when present, typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A composition for selectively heating and/or imaging a cell, or tissue, said composition comprising:
a superparamagnetic nanoparticle attached to a targeting moiety that specifically binds to a biological target comprising said cell or tissue.

2. The composition of claim 1, wherein said superparamagnetic nanoparticle comprises a material that has an electron spin resonance (ESR) Q greater than than 10.

3. The composition of claim 1, wherein said superparamagnetic nanoparticle comprises a material that has an electron spin resonance (ESR) Q ranging from about 100 to about 1000.

4. The composition of claim 1, wherein said superparamagnetic nanoparticle comprises a garnet or a spinel.

5. The composition of claim 4, wherein said superparamagnetic nanoparticle comprises a garnet or a spinel selected from Table 2.

6. The composition of claim 1, wherein said superparamagnetic nanoparticle comprises yttrium ion garnet (YIG).

7. The composition of claim 6, wherein said yttrium ion garnet is substituted with a material selected from the group consisting of aluminum, gallium, indium, and a ferrite.

8. The composition of claim 1, wherein said superparamagnetic nanoparticle comprises gamma-Fe2O3.

9. The composition of claim 6, wherein said yttrium ion garnet is substituted with lithium ferrite.

10. The composition of claim 1, wherein said targeting moiety is directly attached to said superparamagnetic nanoparticle.

11. The composition of claim 1, wherein said targeting moiety is attached to said sperparamagnetic nanoparticle through a linker.

12. The composition of claim 1, wherein said superparamagnetic nanoparticle is attached to a single targeting moiety.

13. The composition of claim 1, wherein said superparamagnetic nanoparticle is attached to a plurality of targeting moieties.

14. The composition of claim 1, wherein a plurality of superparamagnetic nanoparticles are attached to a single targeting moiety.

15. The composition of claim 1, wherein said targeting moiety is selected from the group consisting of a protein, an antibody, a lectin, a saccharide, a vitamin, a steroid, a steroid analogue, a hormone, and a nucleic acid.

16. The composition of claim 1, wherein said cell or tissue is a cancer cell.

17. The composition of claim 16, wherein said targeting moiety is a protein.

18. The composition of claim 16, wherein said targeting moiety is an antibody.

19. The composition of claim 1, wherein said biological target is a cancer marker.

20. The composition of claim 19, wherein said biological target is a cancer marker selected from the group consisting of Caf-1, C-myc, p53, Ki67, Her2, Her4, BRCA1, BRCA2, Lewis Y (Le^{Y}), CA 15-3, G250, HLA-DR cell surface antigen, CEA, CD20, CD22, integrin, cea, 16, EGFr, AR, PSA, and other growth factor receptors.

21. The composition of claim 6, wherein said superparamagnetic nanoparticle has at least one dimension less than about 500 nm.

22. The composition of claim 6, wherein said superparamagnetic nanoparticle has no dimension greater than about 500 nm.

23. The composition of claim 6, wherein said superparamagnetic nanoparticle has at least one dimension less than about 100 nm.

24. A composition for selectively heating or imaging a cell, tissue, or organ, said composition comprising superparamagnetic nanoparticles in a pharmacologically acceptable excipient.

25. The composition of claim 24, wherein said superparamagnetic nanoparticles comprise a material that has electron spin resonance (ESR) Q greater than 10.

26. The composition of claim 24, wherein said superparamagnetic nanoparticles comprise a material that has an electron spin resonance (ESR) Q ranging from about 100 to about 1000.

27. The composition of claim 24, wherein said superparamagnetic nanoparticles comprises a garnet or a spinel.

28. The composition of claim 24, wherein said superparamagnetic nanoparticles comprises a garnet or a spinel selected from Table 2.

29. A method of selectively heating a cell, tissue, or molecule, said method comprising:
contacting said cell, tissue, or molecule with a composition comprising a superparamagnetic nanoparticle attached to a targeting moiety that specifically binds to a biological target comprising said cell, tissue, or molecule; and
heating said superparamagnetic nanoparticle using electron spin resonance.

30. The method of claim 29, wherein said electron spin resonance is at an RF ranging from about RF frequency ranging from 200 to 2,000 MHz MHz.

31. The method of claim 29, wherein said electron spin resonance is at an RF ranging from about 500 to about 1,000 MHz.

32. The method of claim 29, wherein said electron spin resonance is spatially localized by a magnetic field gradient over a region smaller than the region over which the superparamagnetic nanoparticles are distributed.

33. The method of claim 29, wherein said superparamagnetic nanoparticle comprises a material that has an electron spin resonance (ESR) Q greater than 10.

34. The method of claim 29, wherein said superparamagnetic nanoparticle comprises a material that has an electron spin resonance (ESR) Q ranging from about 100 to about 1000.

35. The method of claim 29, wherein said superparamagnetic nanoparticle comprises a garnet or a spinel.

36. The method of claim 35, wherein said superparamagnetic nanoparticle comprises a garnet or a spinel selected from Table 2.

37. The method of claim 29, wherein said superparamagnetic nanoparticle comprises yttrium ion garnet (YIG).

38. The method of claim 37, wherein said yttrium ion garnet is substituted with a material selected from the group consisting of aluminum, gallium, indium, and a ferrite.

39. The method of claim 29, wherein said superparamagnetic nanoparticle comprises gamma-Fe2O3.

40. The method of claim 37, wherein said yttrium ion garnet is substituted with lithium ferrite.

41. The method of claim 29, further comprising imaging said cell, tissue, or molecule using a method selected from the group consisting of thermography, MRI, ESR, and x-ray.

42. The method of claim 29, wherein said targeting moiety is directly attached to said superparamagnetic nanoparticle.

43. The method of claim 29, wherein said targeting moiety is attached to said superparamagnetic nanoparticle through a linker.

44. The method of claim 29, wherein said superparamagnetic nanoparticle is attached to a single targeting moiety.

45. The method of claim 29, wherein said superparamagnetic nanoparticle is attached to a plurality of targeting moieties.

46. The method of claim 29, wherein a plurality of superparamagnetic nanoparticles are attached to a single targeting moiety.

47. The method of claim 29, wherein said targeting moiety is selected from the group consisting of a protein, an antibody, a lectin, a saccharide, a vitamin, a steroid, a steroid analogue, a hormone, and a nucleic acid.

48. The method of claim 29, wherein said cell or tissue is a cancer cell.

49. The method of claim 48, wherein said targeting moiety is a protein.

50. The method of claim 48, wherein said targeting moiety is an antibody.

51. The method of claim 29, wherein said biological target is a cancer marker.

52. The method of claim 51, wherein said biological target is a cancer marker selected from the group consisting of Caf-1, C-myc, p53, Ki67, Her2, Her4, BRCA1, BRCA2, Lewis Y (Le^{Y}), CA 15-3, G250, HLA-DR cell surface antigen, CEA, CD20, CD22, integrin, cea, 16, EGFr, AR, PSA, and other growth factor receptors.

53. The method of claim 29, wherein said superparamagnetic nanoparticle has at least one dimension less than about 500 nm.

54. The method of claim 29, wherein said superparamagnetic nanoparticle has no dimension greater than about 500 nm.

55. The method of claim 29, wherein said superparamagnetic nanoparticle has at least one dimension less than about 100 nm.

56. A method of selectively heating a cell, tissue, or organ, said method comprising:
delivering a plurality of superparamagnetic nanoparticles to a location adjacent to or contacting said cell, tissue, or organ; and
heating said superparamagnetic nanoparticles using electron spin resonance.

57. The method of claim 56, wherein said superparamagnetic nanoparticles are delivered directly into said cell, tissue, or organ.

58. The method of claim 57, wherein said superparamagnetic nanoparticles are delivered directly into said cell, tissue, or organ by injection or via a catheter.

59. The method of claim 57, wherein said superparamagnetic nanoparticles are delivered directly into said cell, tissue, or organ during a surgical procedure.

60. The method of claim 56, wherein said superparamagnetic nanoparticles systemically administered to an organism.

61. The method of claim 56, wherein said electron spin resonance is at an RF ranging from about 200 to about 2,000 MHz.

62. The method of claim 56, wherein said electron spin resonance is at an RF ranging from about 500 to about 1,000 MHz.

63. The method of claim 56, wherein said electron spin resonance is spatially localized by a magnetic field gradient over a region smaller than the region over which the superparamagnetic nanoparticles are distributed.

64. The method of claim 56, wherein said superparamagnetic nanoparticle comprises a material that has an electron spin resonance (ESR) Q greater than 10.

65. The method of claim 56, wherein said superparamagnetic nanoparticle comprises a material that has an electron spin resonance (ESR) Q ranging from about 100 to about 1000.

66. The method of claim 56, wherein said superparamagnetic nanoparticle comprises a garnet or a spinel.

67. The method of claim 66, wherein said superparamagnetic nanoparticle comprises a garnet or a spinel selected from Table 2.

68. The method of claim 66, wherein said superparamagnetic nanoparticle comprises yttrium ion garnet (YIG).

69. The method of claim 68, wherein said yttrium ion garnet is substituted with a material selected from the group consisting of aluminum, gallium, indium, and a ferrite.

70. The method of claim 66, wherein said superparamagnetic nanoparticle comprises gamma-Fe2O3.

71. The method of claim 68, wherein said yttrium ion garnet is substituted with lithium ferrite.

72. The method of claim 56, further comprising imaging said cell, tissue, or molecule using a method selected from the group consisting of thermography, MRI, ESR, and x-ray.

73. The method of claim 56, wherein said cell or tissue is a cancer cell.

74. The method of claim 56, wherein said superparamagnetic nanoparticle has at least one dimension less than about 500 nm.

75. The method of claim 56, wherein said superparamagnetic nanoparticle has no dimension greater than about 500 nm.

76. The method of claim 56, wherein said superparamagnetic nanoparticle has at least one dimension less than about 100 nm.

77. A method of selectively heating or visualizing a cancer cell, said method comprising:
contacting a cancer cell with a chimeric molecule comprising a targeting moiety attached to an epitope tag, wherein said targeting moiety specifically binds to a cancer cell;
contacting said chimeric molecule with a superparamagnetic nanoparticle attached to a binding moiety that specifically binds to said epitope tag thereby associating said superparamagnetic nanoparticle with said cancer cell; and
performing electron spin resonance to heat said superparamagnetic nanoparticle.

78. The method of claim 77, wherein said superparamagnetic nanoparticle comprises a material that has an electron spin resonance (ESR) Q greater than 10.

79. The method of claim 77, wherein said superparamagnetic nanoparticle comprises a material that has an electron spin resonance (ESR) Q ranging from about 100 to about 1000.

80. The method of claim 77, wherein said superparamagnetic nanoparticle comprises a garnet or a spinel.

81. The method of claim 80, wherein said superparamagnetic nanoparticle comprises a garnet or a spinel selected from Table 2.

82. The method of claim 77, wherein said superparamagnetic nanoparticle comprises yttrium ion garnet (YIG).

83. The method of claim 82, wherein said yttrium ion garnet is substituted with a material selected from the group consisting of aluminum, gallium, indium, and a ferrite.

84. The method of claim 77, wherein said superparamagnetic nanoparticle comprises gamma-Fe2O3.

85. The method of claim 82, wherein said yttrium ion garnet is substituted with lithium ferrite.

86. The method of claim 77, further comprising imaging said cell, tissue, or molecule using a method selected from the group consisting of thermography, MRI, ESR, and x-ray.

87. The method of claim 77, wherein said superparamagnetic nanoparticle attached to binding moiety comprises a chelate binding said superparamagnetic nanoparticle attached to said binding moiety.

88. The method of claim 87, wherein said chelate comprises DOTA.

89. The method of claim 77, wherein said epitope tag is an avidin or a biotin.

90. The method of claim 77, wherein said binding moiety is selected from the group consisting of an avidin, a biotin, and an antibody.

91. The method of claim 77, wherein said targeting moiety is directly attached to said epitope tag.

92. The method of claim 77, wherein said targeting moiety is attached to said epitope tag through a linker.

93. The method of claim 77, wherein said targeting moiety is selected from the group consisting of a protein, an antibody, a lectin, a saccharide, a vitamin, a steroid, a steroid analogue, a hormone, a nucleic acid, and an antibody.

94. The method of claim 93, wherein said targeting moiety is a protein.

95. The method of claim 93, wherein said targeting moiety is an antibody.

96. The method of claim 77, wherein said targeting moiety specifically or preferentially binds a cancer marker selected from the group consisting of Caf-1, C-myc, p53, Ki67, Her2, Her4, BRCA1, BRCA2, Lewis Y (Le^{Y}), CA 15-3, G250, HLA-DR cell surface antigen, CEA, CD20, CD22, integrin, cea, 16, EGFr, AR, PSA, and other growth factor receptors.

97. The method of claim 77, wherein said superparamagnetic nanoparticle has at least one dimension less than about 500 nm.

98. The method of claim 77, wherein said superparamagnetic nanoparticle has no dimension greater than about 500 nm.

99. The method of claim 77, wherein said superparamagnetic nanoparticle has at least one dimension less than about 100 nm.

100. A kit for selectively heating or imaging a cell or tissue, said kit comprising:
a container containing a superparamagnetic nanoparticle attached to a targeting moiety that specifically binds to a biological target comprising said cell or tissue.

101. The kit of claim 100, wherein said superparamagnetic nanoparticle comprises a material that has an electron spin resonance (ESR) Q greater than 10.

102. The kit of claim 100, wherein said superparamagnetic nanoparticle comprises a material that has an electron spin resonance (ESR) Q ranging from about 100 to about 1000.

103. The kit of claim 100, wherein said superparamagnetic nanoparticle comprises a garnet or a spinel.

104. The composition of claim 100, wherein said superparamagnetic nanoparticle comprises a garnet or a spinel selected from Table 2.

105. The kit of claim 100, wherein said a superparamagnetic nanoparticle attached to a targeting moiety is in a pharmaceutically acceptable excipient.

106. The kit of claim 100, wherein said kit further comprises instructional materials teaching the use of said superparamagnetic nanoparticles to selectively heat or image a cell or tissue.

107. A kit for selectively heating or imaging a cell or tissue, said kit comprising:
a container containing a superparamagnetic nanoparticle wherein said nanoparticle is derivatized for coupling to a targeting moiety.

108. The kit of claim 107, further comprising a targeting moiety for attaching to said superparamagnetic nanoparticle.

109. The kit of claim 107, wherein said kit further comprises instructional materials teaching the use of said superparamagnetic nanoparticle to selectively heat or image a cell or tissue.

110. The kit of claim 107, wherein said targeting moiety is a moiety that specifically binds to a cancer cell.
